(19) United States
(12) Patent Application Publication
(10) Pub. No.: US 2020/0170641 A1
(43) Pub. Date: Jun. 4, 2020

(54) STOMACH LINING FUNNEL WITH ANASTOMOSIS

(71) Applicant: W. L. Gore & Associates, Inc., Newark, DE (US)

(72) Inventors: Ji Zhang, Newark, DE (US); Sherif A. Eskaros, Elkton, MD (US); Nathan K. Mooney, Elkton, MD (US)

(21) Appl. No.: 16/629,123

(22) PCT Filed: Jul. 7, 2017
(86) PCT No.: PCT/US2017/041069

§ 371 Date: Jan. 7, 2020

(51) Int. Cl. A61B 17/11

(52) U.S. Cl. CPC A61B 17/1114; A61B 2017/00818

(57) ABSTRACT

An anastomosis device includes a collapsible frame forming a funnel with a wide opening narrowing to a central lumen and a membrane covering the collapsible frame. The collapsible frame and the membrane provide a collapsed configuration suitable for endoluminal delivery to a stomach of a patient, and an expanded configuration suitable for lining an internal surface of a gastric wall of the stomach. The anastomosis device further includes an anastomosis component extending from the central lumen of the collapsible frame and being configured to pass through a first hole in the gastric wall and a second hole in a small intestine of the patient and form a sealed connection between the gastric wall and the small intestine. The funnel is configured to substantially close off the pylorus and direct food entering the stomach into the wide opening, through the funnel and into the small intestine via the anastomosis component.

### FIELD

The present disclosure relates to medical devices, and more particularly, but without limitation, to bariatric surgical therapies.

### BACKGROUND

Millions of adults in the United States and elsewhere are obese. Many adults with obesity further suffer from Type 2 Diabetes Mellitus (T2DM) and/or with hypertension. Obesity related disorders, including diabetes, cost the United States and worldwide healthcare systems billions of dollars annually.

Bariatric surgeries, such as vertical sleeve gastrectomy and Roux-en-Y gastric bypass, are effective treatments for both obesity and T2DM. Recent clinical studies demonstrated bariatric surgeries generally provide significantly more excess weight loss in obese patients as compared to lifestyle and medical therapies. Some studies have shown that more than half of bariatric surgery patients also achieve remission of diabetes within a year of surgery.

Safety, early and long-term complications, side effects, and cost associated with bariatric surgeries are some of the major barriers for patients and primary doctors. Often times, patients who qualify for bariatric surgery will forego such intervention in view of one or more of these barriers. Less invasive and more cost-effective treatments could improve patient outcomes.

### SUMMARY

This disclosure includes an anastomosis device suitable for endoscopic delivery and implantation. The anastomosis device includes a funnel configured to cover an internal surface of a gastric wall of a patient and direct food entering the stomach through the funnel and into the small intestine via an anastomosis component, thus restricting nutrient uptake, which can lead to significant weight loss for a patient.

In one example, this disclosure is directed to an anastomosis device comprising a collapsible frame forming a funnel with a wide opening narrowing to a central lumen and a membrane covering the collapsible frame. The collapsible frame and the membrane provide a collapsed configuration suitable for endoluminal delivery to a stomach of a patient, and an expanded configuration suitable for lining an internal surface of a gastric wall of the stomach. The anastomosis device further includes an anastomosis component extending from the central lumen of the collapsible frame and being configured to pass through a first hole in the gastric wall and a second hole in a small intestine of the patient and form a sealed connection between the first hole in the gastric wall and the second hole in the small intestine. The funnel is configured to substantially close off the pylorus and direct food entering the stomach via a patient's esophagus into the wide opening, through the funnel and into the small intestine via the anastomosis component.

In another example, this disclosure is directed to an assembly comprising an endoscopic delivery catheter, and an anastomosis device. The anastomosis device comprises a collapsible frame forming a funnel with a wide opening narrowing to a central lumen, and a membrane covering the collapsible frame. The collapsible frame and the membrane provide a collapsed configuration suitable for endoluminal delivery to a stomach of a patient, and an expanded configuration suitable for lining an internal surface of a gastric wall of the stomach. The anastomosis device further includes an anastomosis component extending from the central lumen of the collapsible frame and being configured to pass through a first hole in the gastric wall and a second hole in a small intestine of the patient and form a sealed connection between the first hole in the gastric wall and the second hole in the small intestine. The funnel is configured to substantially close off the pylorus and direct food entering the stomach via a patient's esophagus into the wide opening, through the funnel and into the small intestine via the anastomosis component. The collapsible frame and the membrane are in the collapsed configuration within a distal end of the endoscopic delivery catheter to facilitate endoscopic delivery and implantation of the anastomosis device within the stomach.

In a further example, this disclosure is directed to a method of implanting an anastomosis device within the stomach of a patient comprising inserting an endoscopic delivery catheter through an esophagus of the patient to locate a distal end of the endoscopic delivery catheter within a stomach of the patient, opening a first hole in a gastric wall of the stomach, opening a second hole in a small intestine of the patient, the second hole being generally coincident with the first hole, and delivering an anastomosis device in a collapsed configuration to the stomach via the endoscopic delivery catheter. The anastomosis device includes a collapsible frame forming a funnel with a wide opening narrowing to a central lumen, a membrane covering the collapsible frame, and an anastomosis component extending from the central lumen of the collapsible frame. The method further includes inserting the anastomosis component through the first hole in the gastric wall and the second hole in the small intestine to form a sealed connection between the first hole in the gastric wall and the second hole in the small intestine, and deploying the anastomosis device from the distal end of the endoscopic delivery catheter to expand the anastomosis device from the collapsed configuration to an expanded configuration and line an internal surface of the gastric wall. Once deployed, the funnel is configured to substantially close off the pylorus and direct food entering the stomach via the esophagus into the wide opening, through the funnel and into the small intestine via the anastomosis component.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIGS. 1A-1B illustrate an anastomosis device including a funnel and an anastomosis component, the anastomosis device being suitable for endoscopic delivery and implantation within the stomach of a patient, according to some examples.

FIGS. 2A-2D are conceptual illustrations of an endoscopic implantation of the anastomosis device of FIGS. 1A-1B, according to some examples.

FIG. 3 is a conceptual illustration of an implanted anastomosis device including a tubular liner configured to extend into the small intestine of the patient, according to some examples.

FIG. 4 illustrates the regions of a lumen apposing metal stent shown in a flat cut pattern.

FIG. 5 illustrates a petal of the lumen apposing metal stent of FIG. 4.

FIG. 6 illustrates a cross section of the lumen apposing metal stent of FIG. 4.

FIGS. 7A and 7B illustrate top and side views of the lumen apposing metal stent of FIG. 4.

FIGS. 8A-8D illustrate incremental deployment of one set of petals of the lumen apposing metal stent of FIG. 4.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

The present disclosure is directed to implantable devices for connecting organ and other tissue structures, for example, to circumvent a conduit or organ blockage, such as by creating a direct passage between organ tissue structures (e.g. connecting a stomach and a portion of a gastrointestinal tract) to create an anastomosis that facilitates material flow therebetween. The devices described herein are endoscopically deployable or deliverable via a catheter and may include self-expanding apposition mechanisms that facilitate a secure connection between the tissue structures (such a connection may also be referred to as a “shunt,” “passageway,” “shunt passageway,” or “tunnel,” for example). Such design features simplify implantation and reduce the likelihood of complications. In some embodiments, the devices provided herein are configured to be removable after implantation. In some examples, the device remains implanted until the body grows a tissue-anastomosis around the device, and then the device is removed. In other embodiments, tissue ingrowth into and/or around the device permanently implants the device, and the device is not removed. The devices described herein can provide alternative treatments for patients who are not suitable candidates for other types of treatments (e.g., such as vertical sleeve gastrectomy and Roux-en-Y gastric bypass) and/or to avoid known complications of other types of treatments.

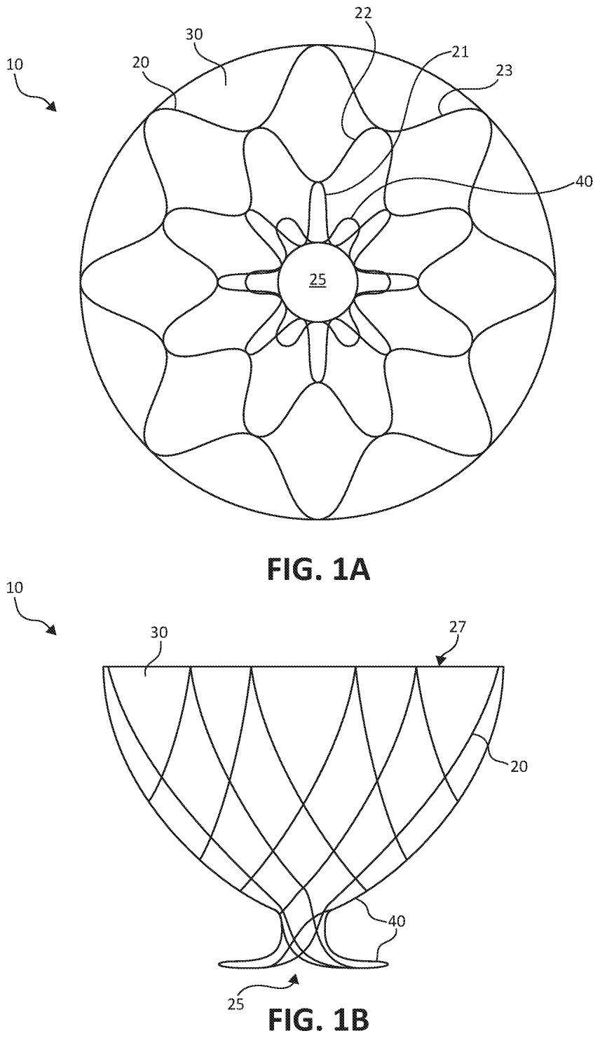

FIGS. 1A-1B illustrate an anastomosis device 10, in accordance with some embodiments provided herein, that can be implanted in a patient to create a fluidic connection between two organs, spaces, tissue structures, conduits, and the like, and combinations thereof. In some examples, the anastomosis device 10 is suitable for endoscopic delivery and implantation within a patient. More specifically, FIG. 1A illustrates a top view of the anastomosis device 10, whereas FIG. 1B illustrates a side view of the anastomosis device 10. The anastomosis device 10 includes a collapsible frame 20, a membrane 30 covering the collapsible frame 20, and an anastomosis component 40.

The collapsible frame 20 forms a funnel with a wide opening 27 narrowing to a central lumen 25. The collapsible frame 20 is formed from one or more elongated elements shaped to form a set of concentric interwoven or interconnected undulating rings 21, 22, 23 radiating from the central lumen 25. In different examples, the undulating rings may represent separate rings or include a single wire in arranged in a coil to form more than one, such as all, of rings 21, 22, 23. The first undulating ring 21 forms a series of peaks and valleys surrounding the central lumen 25. The second undulating ring 22 forms a series of peaks and valleys with the valleys woven through or interconnected with the peaks of the first undulating ring 21. The third undulating ring 23 forms a series of peaks and valleys with the valleys woven through or interconnected with the peaks of the second undulating ring 22. For example, overlapped apices can be held in place with adhesive or the graft material.

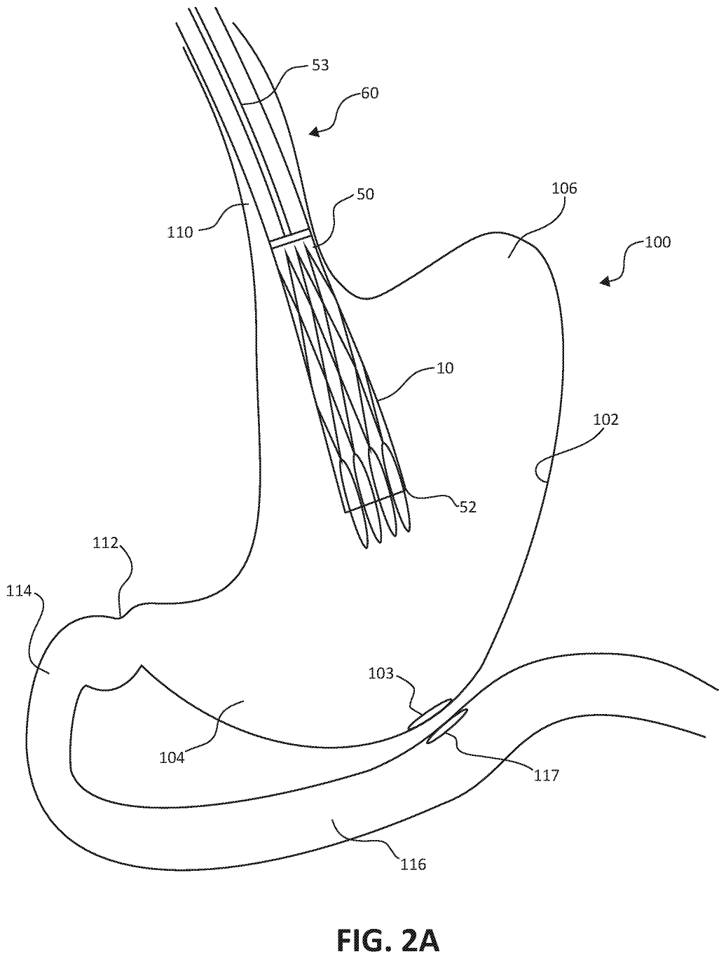

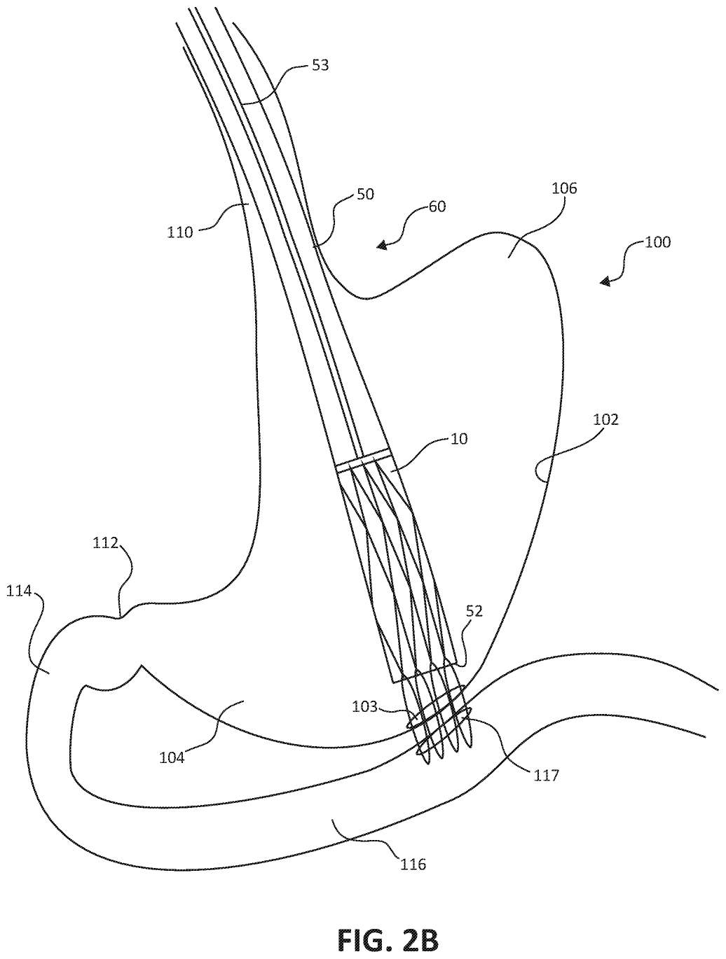

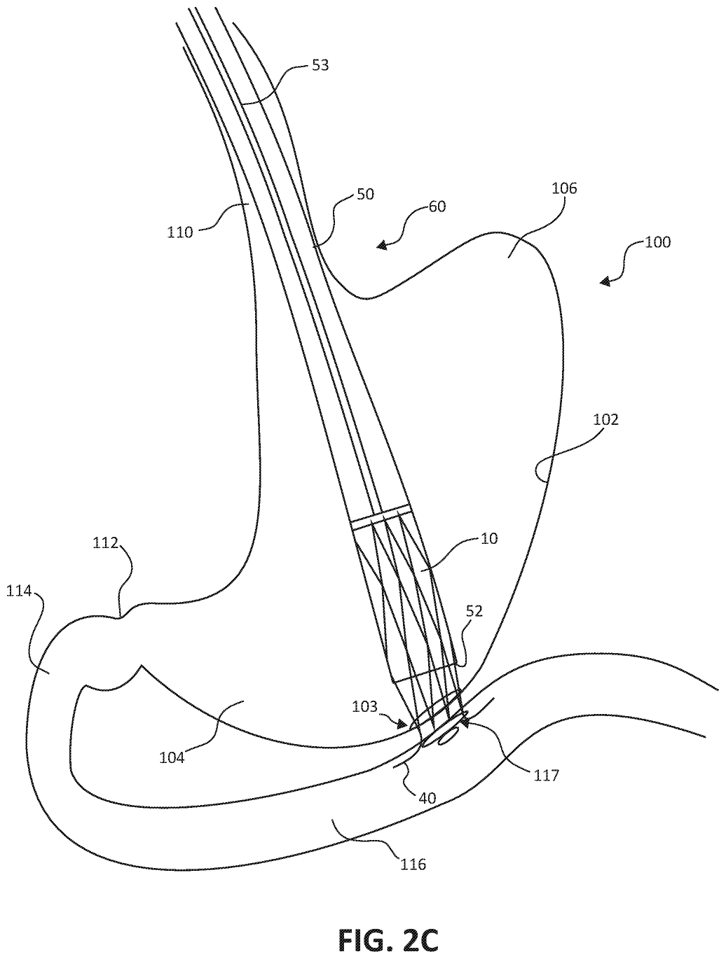

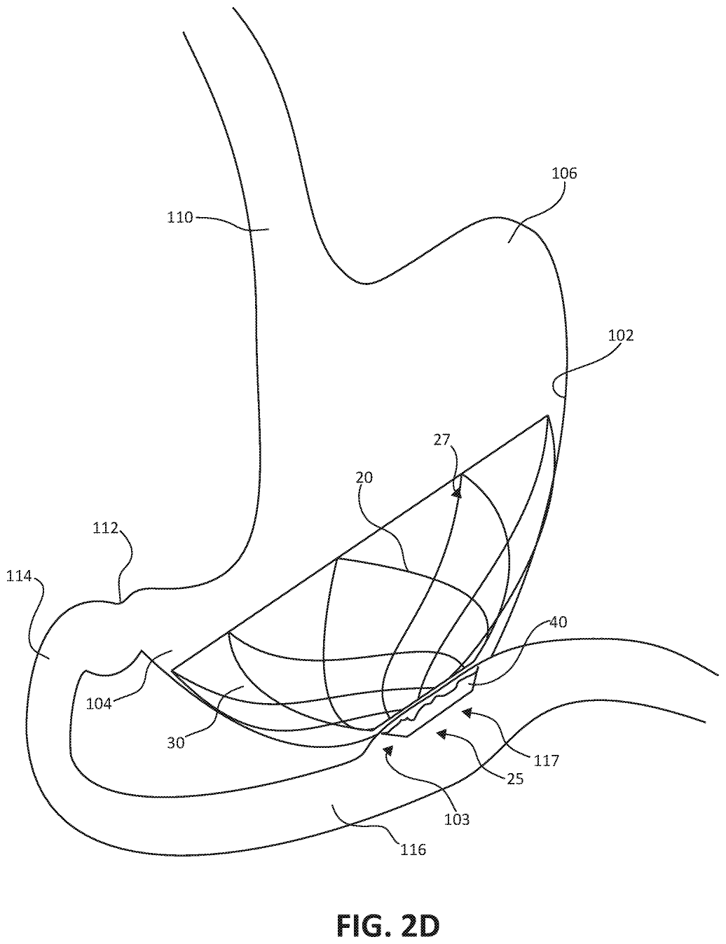

The concentric arrangement of the undulating rings 21, 22, 23 helps provide a collapsed configuration suitable for endoscopic delivery to a stomach of a patient, as shown in FIG. 2A, as well as an expanded configuration suitable for lining an internal surface of a gastric wall of a stomach, as shown in FIG. 2C. The concentric arrangement can additionally or alternatively assist with device flexibility, bendability, conformability, or to achieve other characteristics. In various examples, the collapsible frame 20, when in the expanded configuration is compliant to remain in contact with the internal surface of the gastric wall during peristalsis, for example, or other movement of the stomach wall.

In some examples, the collapsible frame 20 is partially or entirely formed from a metal material, such as a metal wire. In some examples, the collapsible frame 20 is partially or entirely formed from a superelastic material, such as a nitinol wire. Such examples may allow a collapsed configuration suitable for endoscopic delivery through elastic deformation of the expanded configuration. Additionally or alternatively, the collapsible frame 20 can be partially or entirely formed from a cut tube, such as a nitinol tube. Such examples may provide interconnected connections between the undulating rings 21, 22, 23.

The collapsible frame 20 serves as a skeleton to support the membrane 30, and the membrane 30 covers the collapsible frame 20. The membrane 30 is suitable to limit nutrient contact when the anastomosis device 10 is lined against an internal surface of a gastric wall of a stomach. In some examples, the membrane 30 may include or be formed entirely, or primarily (e.g., 80% or greater) from expanded polytetrafluoroethylene (ePTFE). Using ePTFE provides a thin, durable, impermeable material to limit nutrient contact from lined surfaces of the gastric wall. In some examples, the membrane 30 may include elastomer imbibing or a folded structure to allow the membrane 30 to be compliant so as to remain in contact with the internal surface of the gastric wall during peristalsis and other movement of a patient. As implanted within a patient, the funnel formed by the collapsible frame 20 and the membrane 30 is configured to substantially close off the pylorus and direct food entering the stomach via a patient's esophagus into the wide opening 27, through the funnel and into the small intestine via the anastomosis component 40.

In some embodiments, the membrane 30 comprises a fluoropolymer, such as an ePTFE polymer, polytetrafluoroethylene (PTFE) polymer, or polyvinylidene fluoride (PVDF) polymer. In some embodiments, the membrane 30 comprises a polyester, a silicone, a urethane, another biocompatible polymer, polyethylene terephthalate (e.g., Dacron®), copolymers, or combinations thereof.

In some embodiments, the membrane 30 (or portions thereof) is modified by one or more chemical or physical processes that enhance one or more properties of the material. For example, in some embodiments, a hydrophilic coating is applied to the membrane 30 to improve the wettability and echo translucency of the material. In some embodiments, the membrane 30, or portions thereof, is modified with chemical moieties that facilitate one or more of cell attachment, cell migration, cell proliferation, and resistance to or promotion of thrombosis. In some embodiments, the membrane 30, or portions thereof, is modified to resist biofouling. In some embodiments, the membrane 30, or portions thereof, is modified with one or more covalently attached drug substances (e.g., heparin, antibiotics, and the like) or impregnated with the one or more drug substances. The drug substances can be released in situ to promote healing, reduce tissue inflammation, reduce or inhibit infections, and to promote various other therapeutic treatments and outcomes. In some embodiments, the drug substance is a corticosteroid, a human growth factor, an anti-mitotic agent, an antithrombotic agent, a stem cell material, or dexamethasone sodium phosphate, to name some embodiments. In some embodiments, a pharmacological agent is delivered separately from the membrane 30 to the target site to promote tissue healing or tissue growth.

Coatings and treatments may be applied to the membrane 30 before or after the membrane 30 is joined or disposed on the framework of the anastomosis device 10. Additionally, one or both sides of the membrane 30, or portions thereof, may be coated. In some embodiments, certain coatings and/or treatments are applied to portions of the membrane 30 located on some portions of the anastomosis device 10, and other coatings and/or treatments are applied to the material(s) located on other portions of the anastomosis device 10. In some embodiments, a combination of multiple coatings and/or treatments are applied to the membrane 30, or portions thereof. In some embodiments, certain portions of the membrane 30 are left uncoated and/or untreated. In some embodiments, the anastomosis device 10 is fully or partially coated to facilitate or frustrate a biological reaction, such as, but not limited to, cell attachment, cell migration, cell proliferation, and resistance to or promotion of thrombosis.

In some embodiments, a first portion of the membrane 30 is formed of a first material and a second portion of the membrane 30 is formed of a second material that is different than the first material. In some embodiments, the membrane 30 includes multiple layers of materials, which may be the same or different. In some embodiments, portions of the membrane 30 have one or more radiopaque markers attached thereto to enhance in vivo radiographic visualization of the anastomosis device 10, or one or more echogenic areas to enhance ultrasonic visibility.

In some embodiments, one or more portions of the membrane 30 are attached to the framework of the anastomosis device 10, such as the collapsible frame 20 and/or a support structure of the anastomosis component 40. The attachment can be accomplished by a variety of techniques such as, but not limited to, stitching the membrane 30 to the framework of the anastomosis device 10, adhering the membrane 30 to the framework of the anastomosis device 10, laminating multiple layers of the membrane 30 to encompass portions of the elongate members of the anastomosis device 10, using clips or barbs, or laminating multiple layers of the membrane 30 together through openings in the framework of the anastomosis device 10. In some embodiments, the membrane 30 is attached to the framework of the anastomosis device 10 at a series of discrete locations thereby facilitating the flexibility of the framework. In some embodiments, the membrane 30 is loosely attached to the framework of the anastomosis device 10. It is to be appreciated that the membrane 30 may be attached to the framework of the anastomosis device 10 using other techniques or combinations of techniques described herein.

In some embodiments, the framework of the anastomosis device 10 (or portions thereof) is coated with a bonding agent (e.g., fluorinated ethylene propylene or other suitable adhesive) to facilitate attachment of the membrane 30 to the framework. Such adhesives may be applied to the framework using contact coating, powder coating, dip coating, spray coating, or any other appropriate means.

The membrane 30 can adapt to changes in the length and/or diameter of the collapsible frame 20 in a variety of manners. In a first example, the membrane 30 can be elastic such that the membrane 30 can stretch to accommodate changes in the length and/or diameter of the anastomosis device 10. In a second example, the membrane 30 can include slackened material in the low-profile delivery configuration that becomes less slackened or totally unslackened when the anastomosis device 10 is in the expanded configuration. In a third example, the membrane 30 can include folded portions (e.g., pleats) that are folded in the low-profile configuration and less folded or totally unfolded when the anastomosis device 10 is in the expanded configuration. In other embodiments, an axial adjustment member is free of the membrane 30. In some embodiments, combinations of such techniques, and/or other techniques can be used whereby the membrane 30 can adapt to changes in the length and/or diameter of the collapsible frame 20.

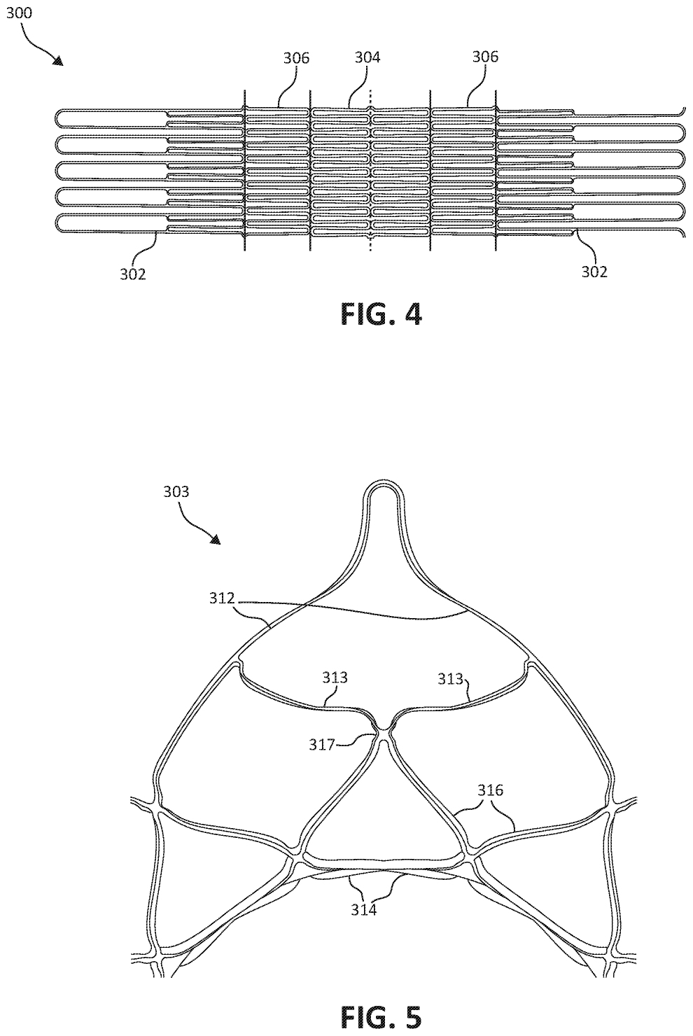

The anastomosis component 40 functions to direct food entering the stomach via a patient's esophagus into the small intestine by sealing a hole in the stomach proximate to central lumen 25 to a corresponding hole in the small intestine. In some examples, the anastomosis component 40 may include a first tissue apposition portion to seal to the hole in the stomach, and a second tissue apposition portion to seal to the hole in the small intestine. The tissue apposition portions of the anastomosis component 40 may include series of petal shaped wire frames surrounding the central lumen 25, although any variety of other configurations of the anastomosis component 40 may serve as suitable alternatives, such as solid petals rather than wire frame petals. An apposition force of the anastomosis component 40 is applied to the external surface of the gastric wall, as described with respect to stent 300, including flanges 302 with petals 303 (FIGS. 4-7B). In contrast to stent 300, the collapsible frame 20 expands much larger on one side, rather than symmetric, although anastomosis component 40 may be functionally the same or similar to stent 300.

Some examples include an optional central portion therebetween, such as barrel 304 of stent 300 (FIGS. 4-7B), providing an extended central lumen 25 that extends longitudinally from a first end of the anastomosis component 40 to a second end of the anastomosis component 40. In any event, central lumen 25 acts as a connection (e.g., a shunt passageway) between the stomach and the intestine, such that the stomach is in fluid communication with the intestines. In some example, the central lumen may be larger than the holes in the stomach and intestinal tissues to provide a slight outward radial force on the holes to aid in sealing, e.g., via an interference fit due to the elasticity of the tissues.

While any number of anastomosis configurations is suitable for adaptation as anastomosis component 40, some of such suitable examples are disclosed in United States Patent Application Publication No. 2015/0313598 by Todd et al., titled ANASTOMOSIS DEVICES.

In some examples, the support structure of the anastomosis component 40 may be formed from a metal material, such as a metal wire. In the same or different examples, the support structure of the anastomosis component 40 may be formed from a superelastic material, such as a nitinol material. Such examples may allow a collapsed configuration suitable for endoscopic delivery through elastic deformation of the expanded configuration. The support structure of the anastomosis component 40 may be formed from a substantially similar material to that of the collapsible frame 20. For example, the collapsible frame 20 and the support structure of the anastomosis component 40 may include a monolithic frame element forming at least a portion of the collapsible frame 20 and the support structure of the anastomosis component 40. In some examples, the collapsible frame 20 and the support structure of the anastomosis component 40 may be formed from a single woven wire, such as a nitinol wire. Alternatively, the collapsible frame 20 and the support structure of the anastomosis component 40 may be formed from a cut tube structure, such as a cut nitinol tube. In further examples, the collapsible frame 20 and the support structure of the anastomosis component 40 may be formed from more than one element including any combination of wire elements, and/or cut tube elements.

In various examples, the membrane 30 may cover the support structure of the anastomosis component 40, or it may not cover the support structure of the anastomosis component 40. In some particular examples, the anastomosis component 40 may be covered in a material that resists ingrowth and adhesion. This may allow the anastomosis device 10 to be removed later without significant trauma to the surrounding tissues of the gastric wall 102.

Suitable materials for the frame elements of the collapsible frame 20 and the support structure of the anastomosis component 40, include a variety of metallic materials including alloys exhibiting shape memory, elastic and super-elastic characteristics. Shape memory refers to the ability of a material to revert to an originally memorized shape after plastic deformation by heating above a critical temperature. Elasticity is the ability of a material to deform under load and return or substantially return to its original shape when the load is released. Most metals will deform elastically up to a small amount of strain. Super-elasticity refers to the ability of a material to deform under strain to much larger degree than typical elastic alloys, without having this deformation become permanent. For example, the super-elastic materials included in the frame elements of some anastomosis device embodiments provided herein are able to withstand a significant amount of bending and flexing and then return or substantially return to the frame's original form without deformation. In some embodiments, suitable elastic materials include various stainless steels which have been physically, chemically, and otherwise treated to produce a high springiness, metal alloys such as cobalt chrome alloys (e.g., ELGILOY™, MP35N, L605), platinum/tungsten alloys. Embodiments of shape memory and super-elastic alloys include the NiTi alloys, ternary shape memory alloys such as NiTiPt, NiTiCo, NiTiCr, or other shape memory alloys such as copper-based shape memory alloys. Additional materials could combine both shape memory and elastic alloys such as a drawn filled tube where the outer layer is constructed of nitinol and the internal core is a radiopaque material such as platinum or tantalum. In such a construct, the outer layer provides the super-elastic properties and the internal core remains elastic due to lower bending stresses.

In some embodiments, the frame elements used to construct the various device examples can be treated in various ways to increase the radiopacity of the devices for enhanced radiographic visualization. In some embodiments, the devices are at least partially a drawn-filled type of NiTi containing a different material at the core, such as a material with enhanced radiopacity. In some embodiments, the devices include a radiopaque cladding or plating. In some embodiments, one or more radiopaque markers are attached to the devices. In some embodiments, the elongate frame elements and/or other portions of the devices provided herein are also visible via ultrasound.

FIGS. 2A-2D illustrate endoscopic implantation of the anastomosis device 10 within the stomach 100 of a patient. The anastomosis device 10 is introduced to the stomach 100 as part of an assembly 60 in a collapsed configuration within an endoscopic delivery catheter 50. The illustrated portion of the patient's anatomy in FIGS. 2A-2D includes the stomach 100, the esophagus 110, the pylorus 112, the duodenum 114, and the jejunum 116 of the patient's small intestine. The stomach 100 includes the gastric wall 102, the antrum 104 and the fundus 106.

As shown in FIG. 2A, the anastomosis device 10 is delivered to the stomach 100 via an endoscopic delivery catheter 50. In some examples, the anastomosis device 10 is carried into the stomach 100 within the distal end 52 of the endoscopic delivery catheter 50. In other examples, the endoscopic delivery catheter 50 may be passed through the esophagus 110 to locate the distal end 52 within the stomach 100 before the anastomosis device 10 is pushed through a central lumen of the endoscopic delivery catheter 50, for example, by first loading the anastomosis device 10 in a proximal end (not shown) of the endoscopic delivery catheter 50 before traversing the length of the central lumen of the endoscopic delivery catheter 50. In such examples, the endoscopic delivery catheter 50 maybe used to facilitate the endoscopic delivery of multiple tools and implants to the stomach 100, such as cameras, surgical tools, and multiple the anastomosis devices 10.

In one exemplary technique of implanting the anastomosis device 10 within the stomach 100, a clinician first inserts the endoscopic delivery catheter 50 through the esophagus 110 to locate the distal end 52 of the endoscopic delivery catheter 50 within the stomach 100. The endoscopic delivery catheter 50 provides access to the stomach 100 for imaging equipment and surgical tools. The clinician then inserts a cutting instrument (not shown) through the endoscopic delivery catheter 50 to a location on an internal surface of the gastric wall 102. The clinician opens a first hole 103 in the gastric wall 102, and a second hole 117 in the small intestine of the patient, such as in the jejunum 116, the second hole 117 being generally coincident with the first hole 103.

The clinician may then withdraw the cutting instrument from the endoscopic delivery catheter 50, and deliver the anastomosis device 10 in a collapsed configuration to the stomach 100 via the endoscopic delivery catheter 50, by pushing the anastomosis device 10 through the central lumen of the endoscopic delivery catheter 50 with the plunger 53, as shown in FIG. 2A.

As shown in FIG. 2B, the clinician may then locate the distal end 52 of the endoscopic delivery catheter 50 proximate the hole 103 and direct the distal end of the anastomosis component 40, which protrudes from the distal end 52 of the endoscopic delivery catheter 50, through the holes 103, 117.

As shown in FIG. 2C, once the distal end of the anastomosis component 40 extends through the holes 103, 117, the clinician may partially deploy the anastomosis device 10 from the distal end 52 of the endoscopic delivery catheter 50. Once deployed, the anastomosis component 40 forms a sealed connection between the first hole 103 in the gastric wall 102 and the second hole 117 in the in the jejunum 116. Next, the clinician may deploy the anastomosis device 10 from the distal end 52 of the endoscopic delivery catheter 50, e.g., by pushing the anastomosis device 10 through the central lumen of endoscopic delivery catheter 50 with the plunger 53.

As shown in FIG. 2D, once deployed, the anastomosis device 10 expands from a collapsed configuration within the central lumen of the endoscopic delivery catheter 50 to an expanded configuration. In the expanded configuration, the collapsible frame 20 and the membrane 30 line an internal surface of the gastric wall 102. In this expanded configuration, the collapsible frame 20 lays flat against the internal surfaces of the gastric wall 102 such that the collapsible frame 20 and the membrane 30 limit nutrient contact from lined portions of the internal surface of the gastric wall 102. For example, in the expanded configuration, the collapsible frame 20 and the membrane 30 may be configured to cover the fundus 106 and a greater curvature of the stomach 100. Such stomach lining may effectively exclude a significant proportion of the hormone producing stomach cells, mimicking a vertical sleeve gastrectomy.

In addition, once anastomosis device 10 is deployed, the funnel of the collapsible frame 20 and the membrane 30 is configured to substantially close off the pylorus 112 and direct food entering the stomach 100 via the esophagus 110 into the wide opening 27, through the funnel and into the small intestine via the anastomosis component 40. The funnel portion of the collapsible frame 20 is of low radial stiffness such that it is compliant and remains in contact with the inner surface of the gastric wall 102 during peristalsis. The funnel portion of the collapsible frame 20 can be sized and shaped to any relevant geometry. In this manner, the anastomosis device 10 can effectively exclude lined portions of the stomach 100 and duodenum 114 and also accelerate food delivery to the jejunum 116, mimicking Roux-en-Y gastric bypass surgery.

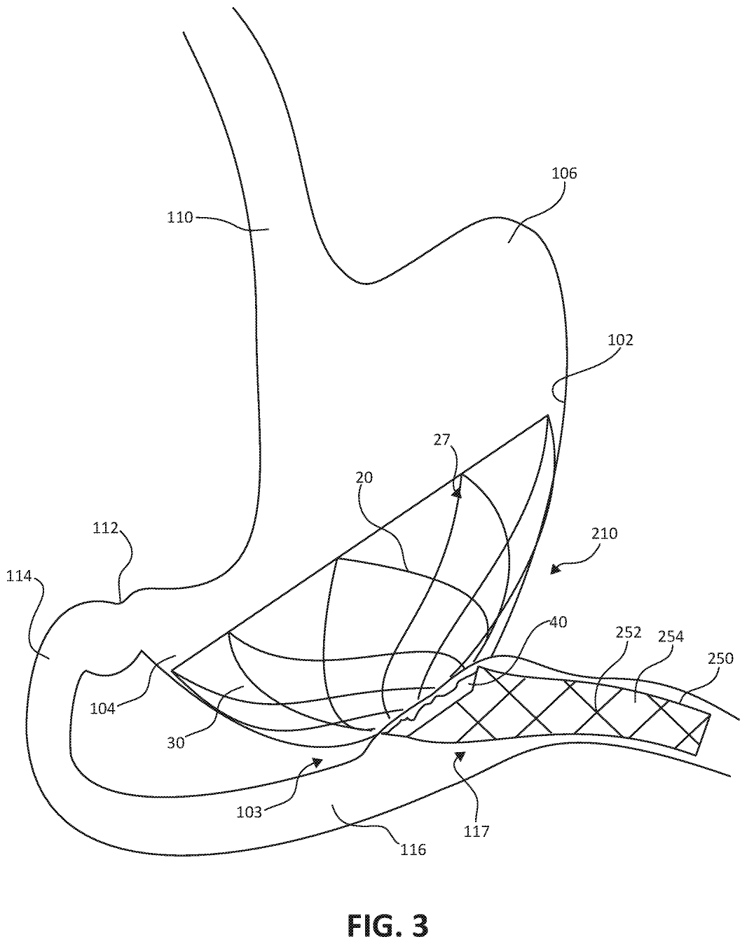

FIG. 3 is conceptual illustration of an implanted anastomosis device 210. The anastomosis device 210 is substantially similar to the anastomosis device 10 and all described variations and equivalents thereof, with the addition of a tubular liner 250. The tubular liner 250 is configured to extend into the jejunum 116 of the small intestine of the patient beyond the anastomosis component 40. For brevity, only the tubular liner 250 is described with respect to anastomosis device 210, as all other elements of the anastomosis device 210 are described with respect to the anastomosis device 10.

The tubular liner 250 forms a central lumen fluidly connected to the lumen 25. The tubular liner 250 is configured to extend from the anastomosis component 40 into the small intestine, such as into the jejunum 116, to line an inner surface of the small intestine to limit nutrient contact from the lined inner surface of the small intestine. This may provide additional efficacy for the patient as compared to the anastomosis device 10 by further limiting portions of the small intestine available for nutrient uptake. In addition, tubular liner 250 may function to help anchor anastomosis device 210 within stomach 100.

The tubular liner 250 includes a frame element 252 and a membrane 254. In some examples, the tubular liner may represent a stent graft. In some examples, the frame element may 252 be an extension of the collapsible frame 20 and/or the support structure of the anastomosis component 40. In other examples, the frame element 252 may be a separate component. In any event, the variations and descriptions above with respect to the collapsible frame 20 and/or the support structure of the anastomosis component 40 are also applicable to frame element 252. As examples, frame element 252 may be formed from a wound wire or individual ring elements or formed from a cut tube. In any of these examples, frame element 252 may be formed from nitinol or stainless steel, and may be either self-expandable, balloon expandable or a combination thereof.

In addition, the membrane 254 may be the same or similar to or even an extension of membrane 30. For example, membrane 254 may comprise a fluoropolymer, such as an ePTFE membrane, or PVDF. In some embodiments, the membrane 30 comprises a polyester, a silicone, a urethane, another biocompatible polymer, polyethylene terephthalate (e.g., Dacron®), copolymers, or combinations thereof.

In some examples, as part of the implantation of the anastomosis device 210, the endoscopic delivery catheter 50 may be directed through holes 103, 117 and into the small intestine to facilitate deployment of tubular liner 250 within the small intestine, such as within the jejunum 116. For example, if tubular liner 250 is self-expandable, endoscopic delivery catheter 50 may function to maintain tubular liner 250 in a collapsed configuration within the small intestine prior to deployment.

FIG. 4 illustrates regions of a lumen opposing metal stent 300 shown in a flat cut pattern. In some examples, the stent 300 may be a self-expanding, covered nitinol stent. In the same or different examples, the stent 300 may be suitable as an implantable internal gallbladder drainage device, or the design of the stent 300 may form the anastomosis component 40 of the anastomosis device 10 or the anastomosis device 210. In various examples, the stent 300 may intended for minimally invasive endoscopic ultrasound (EUS) guided transluminal drainage applications, including internal gallbladder drainage.

The stent 300 includes two flanges 302 connected by a cylindrical barrel 304 with transition regions 305 between the flanges 302 and the flanges 302. In some examples these flanges may correspond to elements of the anastomosis component 40 of the anastomosis device 10 or the anastomosis device 210. Stent 300 may be implanted such that each of the flanges 302 is located on the intra-luminal side of the connected tissues and the barrel 304 spans the combined thickness of both tissue walls. This creates a conduit for contents to pass through the barrel 304.

As shown in FIG. 4, the barrel 304 is defined by the struts that make up the cylindrical portion of the stent 300. The transition regions 306 are include the struts that bend perpendicular to the barrel axis and connect barrel 304 to the petals forming the flanges 302. The flanges 302 are defined by the struts that form the individual petals which collectively form the flanges 302 once shapeset. In some examples, the barrel 304 and the transition regions 306 are symmetric. In the same or different examples, all struts within the barrel 304 and the transition regions 306 may be of equal length and width. In the same or different examples, the flanges 302 may be of equal design or one flange may be larger than the other, or may include additional struts, as with the anastomosis device 10 and the anastomosis device 210. In the same or different examples, petals of the flanges 302 may be offset 1/2n (n=# petals) of the tube circumference such that the each petal will align equally between two of the opposite flange petals.

FIG. 5 illustrates a petal 303 of the flanges 302 of the stent 300. The petal 303 contains both petal struts 312, which define the petal shape, and tether struts 313, which connect roughly midway along the petal struts 312 to the transition struts 316. The tether struts 313 are configured to pull the connected transition strut apex 317 down during crush loading and facilitate a consistent crushed device profile.

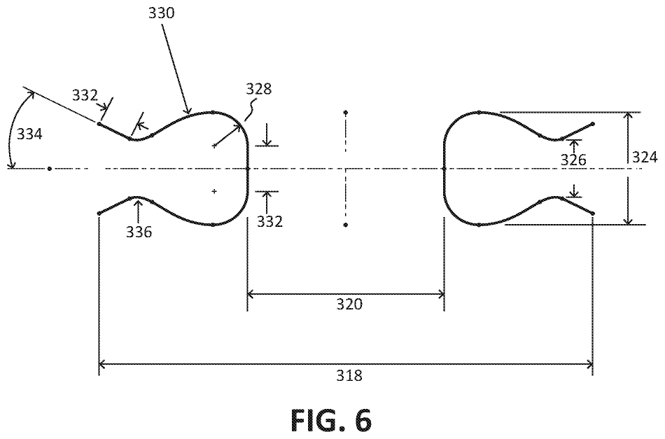

FIG. 6 illustrates a cross section of the stent 300. The cross section of the stent 300 may also represent the profile of tooling used to form the stent 300 to its final shape. Specifically, FIG. 6 illustrates a flange diameter 318, a barrel diameter 320, a barrel flat 322 and a barrel length 324. The barrel length 324 is sized appropriately to accommodate the anticipated combined maximum tissue thickness of the intended treatment range. FIG. 6 further illustrates a petal gap 326, a transition radius 328, a petal radius 330, a petal tip length 332, a petal tip angle 334 and a petal recurve radius 336. The petal gap 326 is less than the barrel length 324, and is generally no larger than a minimum tissue thickness of the intended treatment range, so that the petals 303 are configured to displace outward to accommodate the combined tissue thickness, such as stomach and intestinal tissues in the anastomosis device 10 or the anastomosis device 210 once implanted. This creates strain in the petal 303 and transition struts 316 which results in an apposition force to keep the tissue walls in contact.

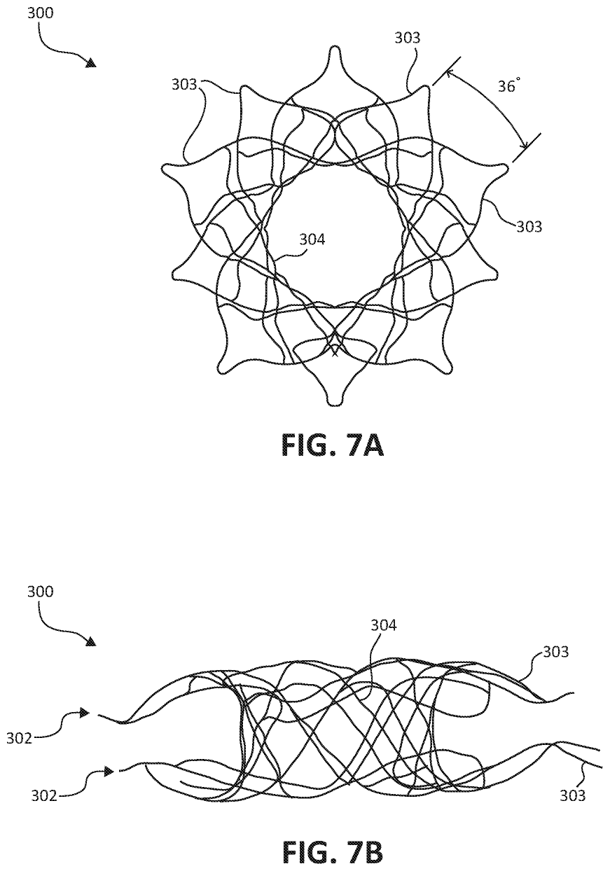

FIGS. 7A and 7B illustrate top and side views of stent 300. FIG. 7A is a front view down the barrel 304. As shown, stent 300 includes five petals 303 per flange 302. Petals are rotationally offset 36 degrees such that they are each centered about two opposing petals. This offset may limit peak pressure points the stent 300 applies to tissue walls between the flanges 302. Additionally, this offset may help balance size-up and crush strain within the stent frame. FIG. 7B is a side view showing the cylindrical barrel 304 and the flanges 302. Note the flange profile is the same as shown in FIG. 6.

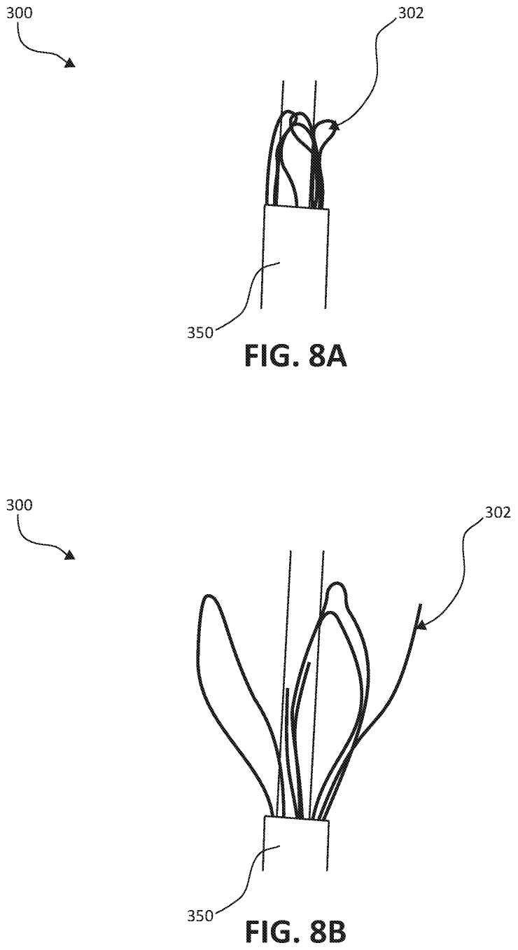

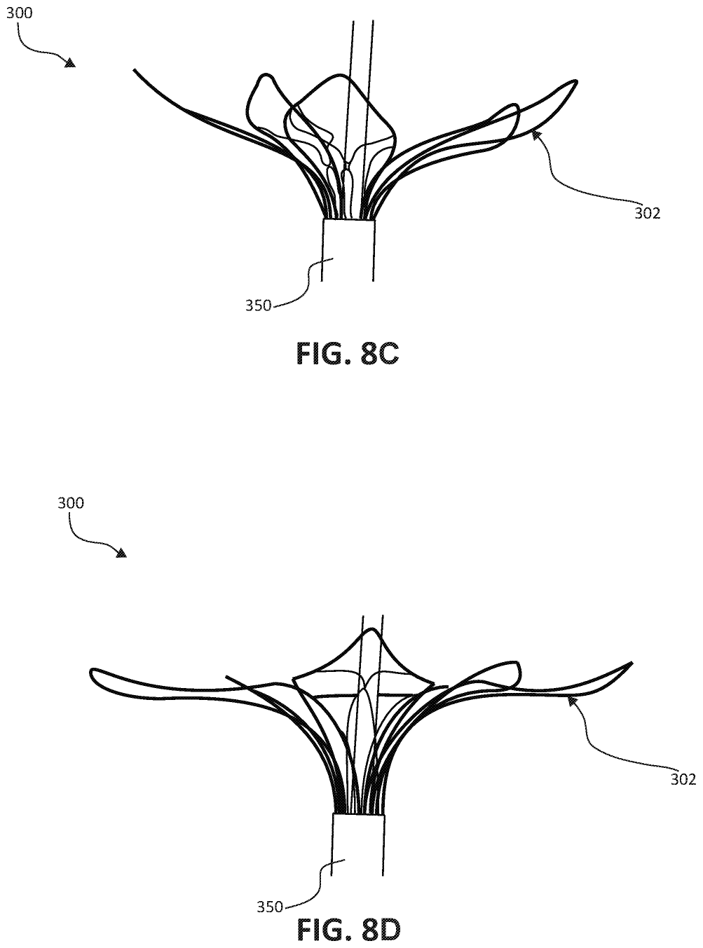

FIGS. 8A-8D illustrate incremental deployment of one flange 302 of the stent 300 from a catheter 350. The transition radius 328 and the petal radius 330 (FIG. 6) are selected facilitate the deployment and in-vivo performance, e.g., by designing for desired apposition forces, as well as allowing for elastic deformation during crush loading. As the stent 300 is bent back against that curvature during crush loading a strain is induced along the length of the petal 303. This strain is released as the stent 300 is unconstrained during deployment. As the stent 300 is incrementally deployed the distal flange 302 opens and spreads while the barrel 304 and proximal region are maintained at the crush profile (FIG. 8D). At this point the stent 300 will resist a traction force applied to it, which allows the two lumens to be pulled into apposition.

Once the two lumens have been pulled into apposition, the barrel and proximal flange are fully deployed (refer to FIG. 7B). Once again the strain induced in the transition radius 328 and the petal radius 330 (FIG. 6) is recovered quickly which forces the proximal flange 302 to snap open and capture the tissue wall. This must occur rapidly because as soon as the stent 300 is fully released it loses contact with the delivery catheter 350 and the traction force the user has applied to provide tissue apposition is lost and the anatomy will return to a near native position.

Once fully deployed, the stent is designed to apply an apposition pressure to both tissue walls. This is created by the strain induced along the petal frame as the petal gap 326 (FIG. 6) is forced larger than its initial value. That displacement is generated by the in-vivo tissue thickness being greater than the petal gap 326. The petals 303 on either flange 302 are directly connected to one another by the transition struts 316 and the barrel struts 314 (FIG. 5), both of which are axially rigid. This allows each petal 303 to directly oppose its opposites on the other flange 302. This equilibrates the total apposition pressure created by both flanges 302 and allows the device to self-center.

In some examples, as previously mentioned the petals 303 may be offset on either flange 302 balance the crush strain the stent 300. This offset may also make the apposition pressure created at the petal tips to be more distributed around the circumference of the flanges 302.

The IGBD stent 300 is one application of a lumen apposing metal stent. However, this disclosure also applies to other uses, including in combination with the anastomosis device 10 or other device designs for any application with a need to divert flow, provide drainage, provide access, anchor, or occlude orifices.

In various examples, this disclosure covers each of following clauses, as well as the claims provided below, although this disclosure is not limited by the listings of clauses and claims.

Clause 1: An anastomosis device comprising: a collapsible frame forming a funnel with a wide opening narrowing to a central lumen; a membrane covering the collapsible frame, the collapsible frame and the membrane providing a collapsed configuration suitable for endoluminal delivery to a stomach of a patient, and an expanded configuration suitable for lining an internal surface of a gastric wall of the stomach; and an anastomosis component extending from the central lumen of the collapsible frame and being configured to pass through a first hole in the gastric wall and a second hole in a small intestine of the patient and form a sealed connection between the first hole in the gastric wall and the second hole in the small intestine, wherein the funnel is configured to substantially close off the pylorus and direct food entering the stomach via a patient's esophagus into the wide opening, through the funnel and into the small intestine via the anastomosis component.

Clause 2: The anastomosis device of clause 1, wherein the anastomosis component comprises a support structure extending from the collapsible frame and being configured to pass through the first hole in the gastric wall and the second hole in the small intestine and lay flat against an internal wall of the small intestine when the anastomosis device is implanted within the patient.

Clause 3: The anastomosis device of clause 2, wherein the membrane covers the support structure of the anastomosis component.

Clause 4: The anastomosis device of clause 1, wherein the collapsible frame and the anastomosis component include a monolithic frame element forming at least a portion of the collapsible frame and a support structure of the anastomosis component.

Clause 5: The anastomosis device of clause 1, wherein the collapsible frame and a support structure of the anastomosis component are formed from a cut tube structure.

Clause 6: The anastomosis device of clause 1, wherein the collapsible frame, when in the expanded configuration, is configured to cover a fundus and a greater curvature of the stomach.

Clause 7: The anastomosis device of clause 1, wherein the collapsible frame, when in the expanded configuration, is configured to limit nutrient contact from lined portions of the internal surface of the gastric wall.

Clause 8: The anastomosis device of clause 1, wherein the second hole in the small intestine of the patient enters a jejunum of the patient.

Clause 9: The anastomosis device of clause 1, wherein the membrane includes expanded polytetrafluoroethylene (ePTFE).

Clause 10: The anastomosis device of clause 1, further comprising a tubular liner configured to extend from the anastomosis component into the small intestine to line an internal surface of the small intestine to limit nutrient contact from the lined internal surface of the small intestine.

Clause 11: The anastomosis device of clause 10, wherein the tubular liner includes a stent graft comprising a frame element and the membrane.

Clause 12: An assembly comprising: an endoscopic delivery catheter; and an anastomosis device comprising: a collapsible frame forming a funnel with a wide opening narrowing to a central lumen; a membrane covering the collapsible frame, the collapsible frame and the membrane providing a collapsed configuration suitable for endoluminal delivery to a stomach of a patient, and an expanded configuration suitable for lining an internal surface of a gastric wall of the stomach; and an anastomosis component extending from the central lumen of the collapsible frame and being configured to pass through a first hole in the gastric wall and a second hole in a small intestine of the patient and form a sealed connection between the first hole in the gastric wall and the second hole in the small intestine, wherein the funnel is configured to substantially close off the pylorus and direct food entering the stomach via a patient's esophagus into the wide opening, through the funnel and into the small intestine via the anastomosis component, wherein the collapsible frame and the membrane are in the collapsed configuration within a distal end of the endoscopic delivery catheter to facilitate endoscopic delivery and implantation of the anastomosis device within the stomach.

Clause 13: The assembly of clause 12, wherein the anastomosis component comprises a support structure extending from the collapsible frame and being configured to pass through the first hole in the gastric wall and the second hole in the small intestine and lay flat against an internal wall of the small intestine when the anastomosis device is implanted within the patient.

Clause 14: The assembly of clause 12, wherein the collapsible frame and the anastomosis component include a monolithic frame element forming at least a portion of the collapsible frame and a support structure of the anastomosis component.

Clause 15: The assembly of clause 12, wherein the collapsible frame, when in the expanded

Clause 16: The assembly of clause 12, wherein the collapsible frame, when in the expanded configuration, is configured to limit nutrient contact from lined portions of the internal surface of the gastric wall.

Clause 17: The assembly of clause 12, wherein the membrane includes expanded polytetrafluoroethylene (ePTFE).

Clause 18: The assembly of clause 12, further comprising a tubular liner configured to extend from the anastomosis component into the small intestine to line an internal surface of the small intestine to limit nutrient contact from the lined internal surface of the small intestine.

Clause 19: A method of implanting an anastomosis device within a stomach of a patient, the method comprising: inserting an endoscopic delivery catheter through an esophagus of the patient to locate a distal end of the endoscopic delivery catheter within the stomach of the patient; opening a first hole in a gastric wall of the stomach; opening a second hole in a small intestine of the patient, the second hole being generally coincident with the first hole; delivering the anastomosis device in a collapsed configuration to the stomach via the endoscopic delivery catheter, wherein the anastomosis device includes: a collapsible frame forming a funnel with a wide opening narrowing to a central lumen; a membrane covering the collapsible frame; and an anastomosis component extending from the central lumen of the collapsible frame; inserting the anastomosis component through the first hole in the gastric wall and the second hole in the small intestine to form a sealed connection between the first hole in the gastric wall and the second hole in the small intestine; and deploying the anastomosis device from the distal end of the endoscopic delivery catheter to expand the anastomosis device from the collapsed configuration to an expanded configuration and line an internal surface of the gastric wall, wherein, once deployed, the funnel is configured to substantially close off the pylorus and direct food entering the stomach via the esophagus into the wide opening, through the funnel and into the small intestine via the anastomosis component.

Clause 20: The method of clause 19, wherein inserting the anastomosis component through the first hole in the gastric wall and the second hole in the small intestine to form the sealed connection comprises: locating a distal portion of a support structure of the anastomosis component through the first hole in the gastric wall and the second hole in the small intestine while the anastomosis device is in the collapsed configuration at least partially proximate the distal end of the endoscopic delivery catheter; and deploying the anastomosis device from the distal end of the endoscopic delivery catheter to allow a transition of the anastomosis device from the collapsed configuration to the expanded configuration such that the support structure of the anastomosis component lays flat against an internal wall of the small intestine and seals the anastomosis component to the internal wall of the small intestine.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An anastomosis device comprising:
a collapsible frame forming a funnel with a wide opening narrowing to a central lumen;
a membrane covering the collapsible frame, the collapsible frame and the membrane providing a collapsed configuration suitable for endoluminal delivery to a stomach of a patient, and an expanded configuration suitable for lining an internal surface of a gastric wall of the stomach; and
an anastomosis component extending from the central lumen of the collapsible frame and being configured to pass through a first hole in the gastric wall and a second hole in a small intestine of the patient and form a sealed connection between the first hole in the gastric wall and the second hole in the small intestine,
wherein the funnel is configured to substantially close off the pylorus and direct food entering the stomach via a patient's esophagus into the wide opening, through the funnel and into the small intestine via the anastomosis component.

2. The anastomosis device of claim 1, wherein the anastomosis component comprises a support structure extending from the collapsible frame and being configured to pass through the first hole in the gastric wall and the second hole in the small intestine and lay flat against an internal wall of the small intestine when the anastomosis device is implanted within the patient.

3. The anastomosis device of claim 2, wherein the membrane covers the support structure of the anastomosis component.

4. The anastomosis device of claim 1, wherein the collapsible frame and the anastomosis component include a monolithic frame element forming at least a portion of the collapsible frame and the support structure of the anastomosis component.

5. The anastomosis device of claim 1, wherein the collapsible frame and the support structure of the anastomosis component are formed from a cut tube structure.

6. The anastomosis device of claim 1, wherein the collapsible frame, when in the expanded configuration, is configured to cover a fundus and a greater curvature of the stomach.

7. The anastomosis device of claim 1, wherein the collapsible frame, when in the expanded configuration, is configured to limit nutrient contact from lined portions of the internal surface of the gastric wall.

8. The anastomosis device of claim 1, wherein the second hole in the small intestine of the patient enters a jejunum of the patient.

9. The anastomosis device of claim 1, wherein the membrane includes expanded polytetrafluoroethylene (ePTFE).

10. The anastomosis device of claim 1, further comprising a tubular liner configured to extend from the anastomosis component into the small intestine to line an internal surface of the small intestine to limit nutrient contact from the lined internal surface of the small intestine.

11. The anastomosis device of claim 10, wherein the tubular liner includes a stent graft comprising a frame element and the membrane.

12. An assembly comprising:
an endoscopic delivery catheter; and
an anastomosis device including:
a collapsible frame forming a funnel with a wide opening narrowing to a central lumen;
a membrane covering the collapsible frame, the collapsible frame and the membrane providing a collapsed configuration suitable for endoluminal delivery to a stomach of a patient, and an expanded configuration suitable for lining an internal surface of a gastric wall of the stomach; and
an anastomosis component extending from the central lumen of the collapsible frame and being configured to pass through a first hole in the gastric wall and a second hole in a small intestine of the patient and form a sealed connection between the first hole in the gastric wall and the second hole in the small intestine,
wherein the funnel is configured to substantially close off the pylorus and direct food entering the stomach via a patient's esophagus into the wide opening, through the funnel and into the small intestine via the anastomosis component.

13. The assembly of claim 12, further comprising a plunger configured to push the anastomosis device out a distal end of the endoscopic delivery catheter to facilitate deployment of the anastomosis device within a patient.

14. A method of implanting an anastomosis device claim 1 within the stomach of a patient, the method comprising:
inserting an endoscopic delivery catheter through an esophagus of the patient to locate a distal end of the endoscopic delivery catheter within the stomach of the patient;
opening a first hole in a gastric wall of the stomach;
opening a second hole in a small intestine of the patient, the second hole being generally coincident with the first hole;
delivering an anastomosis device in a collapsed configuration to the stomach via the endoscopic delivery catheter, the anastomosis device including a collapsible frame forming a funnel with a wide opening narrowing to a central lumen; a membrane covering the collapsible frame, the collapsible frame and the membrane providing a collapsed configuration suitable for endoluminal delivery to a stomach of a patient, and an expanded configuration suitable for lining an internal surface of a gastric wall of the stomach; and an anastomosis component extending from the central lumen of the collapsible frame and being configured to pass through a first hole in the gastric wall and a second hole in a small intestine of the patient and form a sealed connection between the first hole in the gastric wall and the second hole in the small intestine;
inserting the anastomosis component through the first hole in the gastric wall and the second hole in the small intestine to form a sealed connection between the first hole in the gastric wall and the second hole in the small intestine; and
deploying the anastomosis device from the distal end of the endoscopic delivery catheter to expand the anastomosis device from the collapsed configuration to an expanded configuration and line an internal surface of the gastric wall,
wherein, once deployed, the funnel is configured to substantially close off the pylorus and direct food entering the stomach via the esophagus into the wide opening, through the funnel and into the small intestine via the anastomosis component.

15. The method of claim 14, wherein inserting the anastomosis component through the first hole in the gastric wall and the second hole in the small intestine to form the sealed connection comprises:
locating a distal portion of a support structure of the anastomosis component through the first hole in the gastric wall and the second hole in the small intestine while the anastomosis device is in the collapsed configuration at least partially proximate the distal end of the endoscopic delivery catheter; and
deploying the anastomosis device from the distal end of the endoscopic delivery catheter to allow the transition of the anastomosis device from the collapsed configuration to the expanded configuration such that the support structure of the anastomosis component lays flat against an internal wall of the small intestine and seals the anastomosis component to the internal wall of the small intestine.
